(19) United States
(12) Patent Application Publication
(10) Pub. No.: US 2025/0255635 A1
(43) Pub. Date: Aug. 14, 2025

(54) SYSTEMS AND METHODS FOR PERCUTANEOUS TREATMENT AND DEBRIDEMENT OF INFECTIONS

(71) Applicant: VANDERBILT UNIVERSITY, Nashville, TN (US)

(72) Inventor: Raymond Joseph Gardocki, Nashville, TN (US)

(21) Appl. No.: 19/043,654

(22) Filed: Feb. 3, 2025

Related U.S. Application Data
(60) Provisional application No. 63/551,634, filed on Feb. 9, 2024.

(51) Int. Cl. A61B 17/32; A61B 17/00; A61B 90/00

(52) U.S. Cl. CPC A61B 17/320068; A61B 17/320016; A61B 90/39; A61B 2017/00761; A61B 2017/320004; A61B 2017/32007; A61B 2090/3966; A61B 2217/005; A61B 2217/007

(57) ABSTRACT

The present disclosure provides for devices, systems and methods of debriding and treating abscesses and infections affecting a cavity, bone, joint, or other area of the body. The present disclosure provides for devices, systems, and methods described herein that allows percutaneous or endoscopic placement (ultra-minimally invasive) of at least a delivery tube and at least an evacuation tube (e.g., using a catheter) to allow for debridement and treatment of spine infections, septic joints, abscess cavities and the like with a greatly reduced morbidity compared to an open surgical approach.

### CROSS-REFERENCE TO RELATED APPLICATIONS

This application claims the benefit of U.S. Provisional Application entitled “SYSTEMS AND METHODS FOR PERCUTANEOUS TREATMENT AND DEBRIDEMENT OF INFECTIONS” and having Ser. No. 63/551,634 filed on Feb. 9, 2024, which is incorporated herein by reference in its entirety.

### BACKGROUND

Bacterial infections are a costly problem for both patients and treatment facilities. In some situations, bacterial infections can develop in a cavity or around a bone or joint, where the infection is isolated from the patient's bloodstream. Traditional treatment methods for these infections involve high dosages of antibiotics and can lead to the development of antibiotic-resistant bacteria.

### SUMMARY

In accordance with the purpose(s) of this disclosure, as embodied and broadly described herein, the disclosure, in various aspects, relates to percutaneous devices and systems for debridement and treatment of infections (e.g., bacterial infections) and methods of use thereof.

Embodiments of the present disclosure provides for a device or a system that includes at least a delivery tube and at least an evacuation tube, and an ultrasonic generator configured to cause ultrasonic waves to be emitted from a distal end of the catheter. In an aspect, the delivery tube and the evacuation tube are part of a catheter or are part of different catheters.

The present disclosure provides for methods of using a debridement device or system (as those described above or herein), comprising: introducing at least a delivery tube and at least an evacuation tube into an infection site; administering fluid to the infection site via the delivery tube; applying ultrasonic waves to the infection site using an ultrasonic generator; and clearing debris from the infection site via the evacuation tube.

The present disclosure also provides for methods of using a debridement device or system, such as the one described above and herein, that can comprise the steps of introducing at least a delivery tube and at least an evacuation tube (or a catheter including these) into an abscess; administering fluid to the abscess via the delivery tube of the catheter; applying ultrasonic waves to the abscess using an ultrasonic generator; and clearing debris from the abscess via the evacuation tube.

Other systems, methods, devices, features, and advantages of the devices and methods will be or become apparent to one with skill in the art upon examination of the following drawings and detailed description. It is intended that all such additional systems, methods, devices, features, and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is an illustration of a catheter according to various embodiments of the present disclosure.

FIG. 2 is an illustration of a system including the catheter of FIG. 1 according to various embodiments of the present disclosure.

FIG. 3 is a flowchart depicting a method implemented using the system of FIG. 2 according to various embodiments of the present disclosure.

### DETAILED DESCRIPTION

The present disclosure provides for various approaches for the percutaneous treatment and debridement of infections using devices or systems can include one or more tubes (e.g., catheters for each tube, a catheter including the tubes) and an ultrasonic generator.

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be conducted in the order of events recited or in any other order that is logically possible.

Embodiments of the present disclosure will employ, unless otherwise indicated, biomedical engineering, mechanical engineering, medical techniques and the like, which are within the skill of the art. Such techniques are explained fully in the literature.

The following examples are put forth to provide those of ordinary skill in the art with a complete disclosure and description of how to perform the methods and use the compositions and compounds disclosed and claimed herein. Efforts have been made to ensure accuracy with respect to numbers (e.g., amounts, measurements, etc.), but some errors and deviations should be accounted for.

Before the embodiments of the present disclosure are described in detail, it is to be understood that, unless otherwise indicated, the present disclosure is not limited to particular materials, machines, computing processes, or the like, as such can vary. It is also to be understood that the terminology used herein is for purposes of describing particular embodiments only and is not intended to be limiting. It is also possible in the present disclosure that steps can be executed in different sequence where this is logically possible.

### Discussion

Disclosed are various approaches for the percutaneous treatment and debridement of infections using a device or a system that includes at least a delivery tube and at least an evacuation tube (e.g., both comprised in a catheter) and an ultrasonic generator. Currently, spine infections, septic joints (both with and without implants), abscesses, and other infections are treated with open surgical debridement, which comes with the associated morbidity of an open surgical approach as well as that for systemic antibiotics. Often, these infections are poorly vascularized centrally, which is also where the highest bacterial load resides. Traditional treatment methods use intravenous (IV) antibiotics, which are circulated through the vasculature, having little impact on the poorly vascularized infection site. Thus, patients are often exposed to large amounts of systemic antibiotics for prolonged periods of time while attempting to treat the infection site. This can lead to the development of antibiotic-resistant bacteria, exacerbating the problem, as well as other problems.

The present disclosure provides for devices, systems and methods of debriding and treating isolated abscesses and infections affecting a cavity, bone, joint, or other area of the body. Aspects of the present disclosure allow for percutaneous or endoscopic placement (ultra-minimally invasive) (e.g., in one or more incisions) of at least a delivery tube and at least an evacuation tube (e.g., using one or more catheters) to allow for debridement and treatment of spine infections, septic joints, abscess cavities and the like with a greatly reduced morbidity compared to an open surgical approach. In an aspect, the devices, systems, and methods can also result in a more effective delivery of antibiotics to the space where they are most needed. This will maximize the antibiotic concentration in the best possible location while simultaneously decreasing systemic absorption leading to less systemic toxicity. The goal is to treat spine infections, septic joints, and abscess cavities quicker, with reduced morbidity and increased effectiveness all while lowering the total cost of treatment. The ability to increase antibiotic concentration to well above the mean inhibitory concentration at the center of the infection with less systemic absorption would also decrease the risk of creating antibiotic resistant bacteria.

Accordingly, various embodiments of the present disclosure are directed to a device or a system having three primary components: an inflow tube (also referred to as the “delivery tube”) and an outflow tube (also referred to as the “evacuation tube”) to apply and drain antibiotics, and a third component which would allow ultrasonic vibration energy to be applied to the patient, for example to the center of the abscess cavity, to debride the infection from the inside. In a particular aspect, the device or system has an inflow catheter (e.g., a first catheter including the inflow tube) and an outflow catheter (e.g., a second catheter including the outflow tube) to apply and drain antibiotics, and a third component which would allow ultrasonic vibration energy to the patient to debride the infection from the inside. In some embodiments the outflow tube can include multiple fenestrations or fluid channels to prevent clogging. In an aspect, inflow tube and the outflow tube can have different dimension (e.g., length, internal diameter, external diameter) In some embodiments, this third component can comprise an ultrasonic generator outside the body with a valve-like device at the catheter tip that allows application of the ultrasonic energy directly to the area of infection, such as a center of the abscess using the first catheter, second catheter or a third catheter (e.g., including a wire that propagate the ultrasonic energy). In an aspect, the inflow tube and the outflow tube can with within a single catheter. According to various examples, an ultrasonic generator can use the inflow catheter to transfer the energy (e.g., using a wire within the inflow catheter to propagate the ultrasonic energy) to the infection. The mechanism of debridement would be similar to that of a jewelry cleaner or ultrasonic clothes washer with the ultrasonic energy being transferred through the liquid antibiotics or saline to debride the dead and infected tissue from within. Then, an antibiotic can be injected and drawn into the cavity with pressure. In some embodiments, suction can be used to control outflow of fluid from area of the infection (e.g., the abscess). In some embodiments, the energy of the ultrasonic generator can be used to push the fluid into the cavity with the aid of check valves to control the total pressure within area, for example, the abscess cavity. The technique for application can involve direct endoscopic or arthroscopic debridement of the area of the infection, abscess or joint, with placement of the catheter(s) through the scope or with radiographically guided percutaneous insertion which could eliminate the surgical cost and risks. In some instances, this system allows for placing the device and sending a patient home with continuous gentle debridement and antibiotic application to decrease length of hospital stay which in turn, can significantly reduce costs for the treatment of severe spine, joint, and abscess infections. In addition, the catheter(s) can be coated with an antibacterial coating.

In addition, the devices, systems and methods described herein provide for the ability to continuously debride an infected cavity or joint space with ultrasonic energy. This can break up the glycocalyx that bacteria form when attached to metallic implants which prevents the antibiotics from reaching the bacteria. Treating prosthetic joint infections in this manner without implant removal results in immense cost savings and decreased morbidity.

In the following discussion, a general description of the system and its components is provided, followed by a discussion of the operation of the same. Although the following discussion provides illustrative examples of the operation of various components of the present disclosure, the use of the following illustrative examples does not exclude other implementations that are consistent with the principles disclosed by the following illustrative examples.

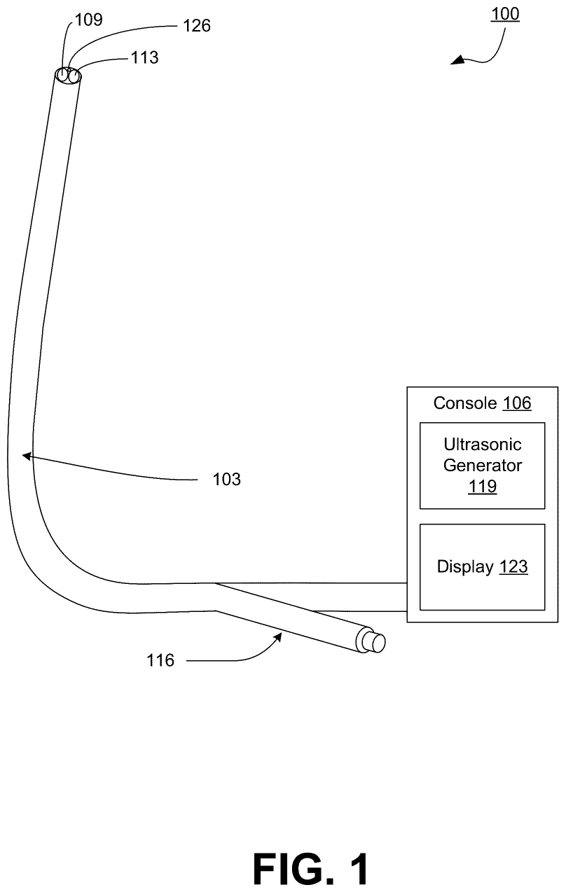

With reference to FIG. 1, shown is a catheter system 100 according to various embodiments. The catheter system 100 can include the catheter 103 and a console 106. The catheter 103 can include a delivery tube 109 and an evacuation tube 113 as well as one or more ports 116 (i.e., guidewire ports, instrumentation ports, inflation ports, injection ports, etc.). The catheter 103 can be configured for positioning within an infection site, such as a cavity, an abscess, or an infected bone or joint. The catheter 103 can be made of a flexible material such as latex, silicone, Teflon, plastic, rubber, a biocompatible material, etc., and can be sized to fit within a minimally invasive entry site near the infection (e.g., an abscess, implant, etc.). In some embodiments the catheter cab be coated with an antibacterial coating either currently available or yet to be developed. In some embodiments, the catheter 103 has an outer diameter near the range of approximately 3 to 15 or 5 to 7 millimeters. In some embodiments, the catheter 103 comprises a pigtail catheter to prevent the distal end from coming out of a patient during a procedure. In an aspect, a suture can secure the device to the area in the body to secure the device to the patient.

As shown in FIG. 1, the catheter 103 can be connected to a console 106, which can include an ultrasonic generator 119 and a display 123. In some embodiments, the console 106 can be configured to control the flow of fluid(s) within the catheter system 100. The console 106 can comprise one or more computing devices. The console 106 may include a processor-based system such as a computer system. In many embodiments, the console 106 can be a specialized computing device made specifically for controlling the catheter system 100. Such a computer system can be embodied in the form of a mobile computing device (e.g., laptop computer, tablet computer system, smartphone, web pads, portable game consoles, and similar devices), a robotic control console, or other devices with like capability. In some embodiments, the console 106 can be configured to execute a control application to control the various different features of the catheter system 100.

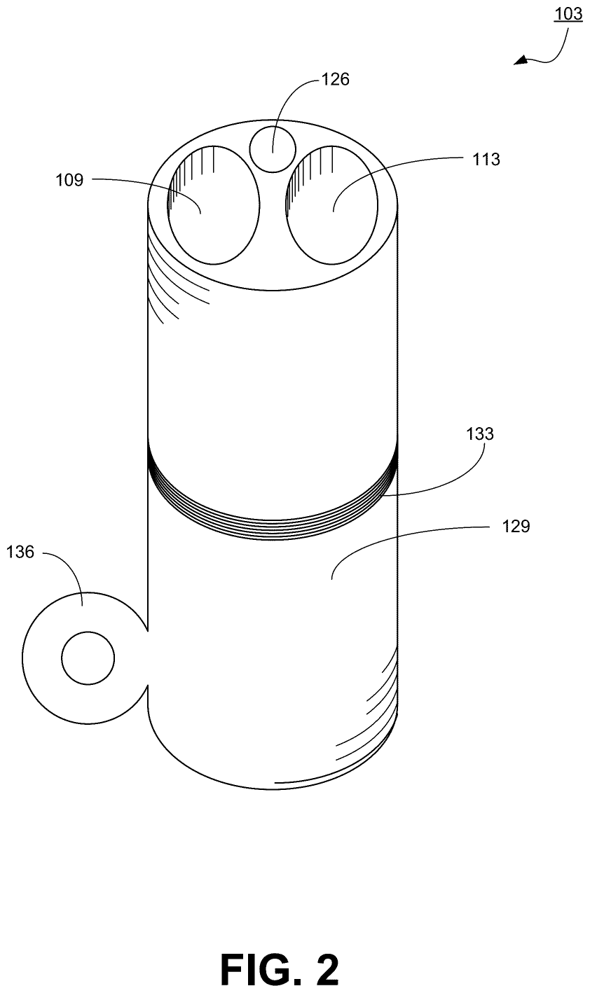

The console 106 can include an ultrasonic generator 119. The ultrasonic generator 119 can be a system for converting electricity into ultrasonic waves using a transducer. The transducer can comprise a piezoelectric transducer, a capacitive transducer, or other type of suitable transducer. The ultrasonic generator 119 can be coupled to the catheter 103 such that ultrasonic waves are emitted from the distal end of the catheter 103. In some embodiments, the transducer of the ultrasonic generator 119 is located at the distal end of the catheter 103 and is electrically coupled to the console 106. In some embodiments, the transducer is located within the console and produces ultrasonic waves which travel through the catheter 103 to the distal end. The ultrasonic waves can travel through the catheter 103 via a wire 126 (see FIG. 2). In some examples, the ultrasonic waves can travel through the catheter 103 via fluid in the delivery tube 109. In some embodiments, the ultrasonic energy delivery is through a catheter separate from catheter 103.

The console 106 can include one or more displays 123, such as liquid crystal displays (LCDs), gas plasma-based flat panel displays, organic light emitting diode (OLED) displays, electrophoretic ink (“E-ink”) displays, projectors, or other types of display devices. In some instances, the display 123 can be a component of the console 106 or can be connected to the console 106 through a wired or wireless connection. In some embodiments, the display 123 is controlled by a display application. The console 106 may be configured to execute the display application. Various data can be shown on the display 123, such as sensor readings, number of flush cycles to be executed, whether the ultrasonic generator 119 is running, warnings, notifications, or other data and communications.

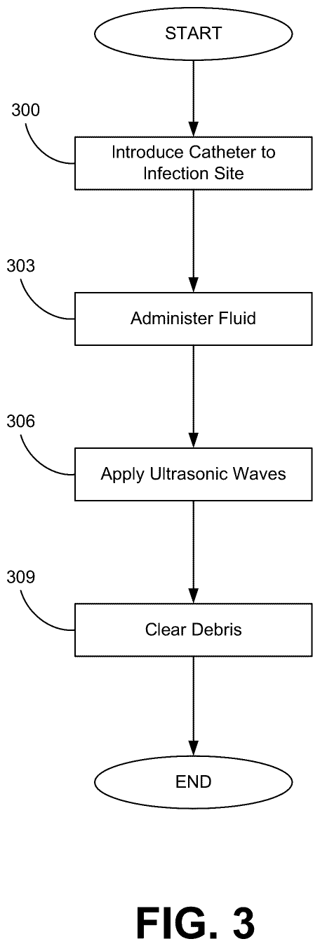

While FIG. 1 is directed to a single catheter including the delivery tube 109 and an evacuation tube 113, a similar design can be employed with the delivery tube and an evacuation tube are each in separate catheters (e.g., a first catheter and a second catheter), where the delivery tube and an evacuation tube are in communication with a console (such as console 106 including the ultrasonic generator 119 and display 123). In addition, the ultrasonic energy can be propagated using the first catheter, the second catheter, or a third catheter. In this regard, all features described in reference to FIG. 1 (as well as FIGS. 2 and 3), also apply to other embodiments described herein.

An example of the operation of the catheter system 100 of FIG. 1 is as follows. The catheter system 100 of FIG. 1 can be operated to deliver a fluid to an infection site in a patient. First, the catheter 103 can be introduced to an infection site. Then, fluid can be administered through the catheter 103 to the infection site. In some embodiments, the fluid can be inserted into the catheter system 100 via a side port 116 on the catheter 103. The fluid can then be administered to the infection site by way of the delivery tube 109 of the catheter 103. The fluid administered to the infection site can be antibiotics, normal saline, hypertonic saline. Or other fluid for flushing or treating an infection site. In some embodiments, the ultrasonic generator 119 can be activated to produce ultrasonic waves at the distal end of the catheter 103. These ultrasonic waves can be applied to fill the infection site and perform debridement. Next, the fluid and debris from the infection site can be cleared using the evacuation tube 113. The evacuation tube 113 can clear the fluid from the infection site using a check valve to control pressure, suction from a vacuum system, passive draining, or other methods of clearing the fluid from the site. According to various examples, this process is repeated until the infection site has been thoroughly cleaned and treated. As stated herein, in some embodiments the delivery tube and the evacuation tube are in separate catheters.

Moving next to FIG. 2, shown is an example of the distal end of the catheter 103 from FIG. 1. In some embodiments, the catheter 103 includes a delivery tube 109 and an evacuation tube 113. As shown in FIG. 2, the delivery tube 109 and the evacuation tube 113 can comprise two lumens which extend along the length of the catheter 103 from a proximal end (not shown) to the distal end. However, in some embodiments, the delivery tube 109 and the evacuation tube 113 are two separate catheter 103 tubes which are connected together by an outer shell 129 of a similar material. In some embodiments, the delivery tube 109 and the evacuation tube 113 are separate tubes. In some embodiments, the delivery tube 109 and the evacuation tube 113 have different terminal lengths. In some embodiments, the delivery tube 109 and the evacuation tube 113 can each have an internal diameter near the range of approximately 1.5 to 12 or 1.5 to 6 millimeters. According to various examples, the delivery tube 109 and the evacuation tube 113 extend from the distal end of the catheter 103 to different respective receptacles. The delivery tube 109 can extend from the distal end of the catheter 103 to a receptacle housing an antibiotic. The delivery tube 109 can extend from the distal end of the catheter 103 to a receptacle housing normal saline or hypertonic saline. In some embodiments, the delivery tube 109 extends from the distal end of the catheter to an injection port 116 where a fluid can be injected. Similarly, the evacuation tube 113 can extend from the distal end of the catheter 103 to a receptacle for waste products which will be removed from an infection site. In some embodiments, the evacuation tube 113 can extend from the distal end of the catheter 103 to a vacuum which provides suction for clearing waste products from the infection site.

The catheter 103 can include a wire 126 for transmitting ultrasonic waves from the ultrasonic generator 119 to the infection site. The wire 126 can be comprised of a metal, conductive polymer, or other material capable of transmitting ultrasonic waves along the catheter 103. The wire 126 can extend from the ultrasonic generator 119 to the distal end of the catheter 103. In some embodiments, the transducer of the ultrasonic generator 119 is located at the distal end of the catheter 103, and no wire 126 is necessary. In some examples, the ultrasonic waves can travel through the catheter 103 via fluid in the catheter 103. As stated herein, the wire can be within the first catheter, the second catheter, and/or the third catheter.

In some embodiments, the catheter 103 can further include one or more depth gauges 133. As shown in FIG. 2, a depth gauge 133 can be a marking on the outer shell 129 of the catheter 103 which is set at a specific distance from the distal end of the catheter 103. The catheter 103 can include one or more depth gauges 133 at progressively further distances from the distal end in order to indicate to a user how far the catheter 103 extends within a body. In some embodiments, the depth gauge 133 can comprise another form of measuring the distance from the distal end of the catheter 103. In some embodiments, the catheter 103 further includes a radiopaque marker disposed at the distal end of the catheter 103 so that the end of the catheter 103 can be detected with medical imaging techniques.

The catheter 103 of FIG. 2 further includes a hoop 136 disposed on the outer shell 129 of the catheter 103. While depicted as a ring-shaped hoop, the hoop 136 can be any shape which is configured to receive a stitch or suture. The hoop 136 can be stitched to a portion of a patient's skin to secure the catheter 103 at an insertion site. In some embodiments, the hoop 136 has a hole in the center to receive the stitch or suture. In some embodiments, the hoop 136 comprises a thin material without a hole, where the thin material can be easily punctured using a suture needle.

Next, at FIG. 3, shown is a flowchart that provides one example of the method of using the catheter system 100 as described above. The flowchart of FIG. 3 provides merely an example of the many different types of functional arrangements that can be employed to implement the operation of the method.

Beginning with block 300, the catheter(s) of the catheter system can be introduced into an infection site, or abscess, via an entry site in a patient. Then, at block 303, fluid can be administered to the infection site via the delivery tube of the catheter(s). Fluid can be administered through an injection port on the catheter which connects to the delivery tube within the catheter(s). In some embodiments, the catheter(s) can be pressurized to drive fluid through the delivery tube. The fluid administered to the infection site can include an antibiotic, normal saline, hypertonic saline, or another solution.

Next, at block 306, ultrasonic waves can be applied to the infection site using an ultrasonic generator. The ultrasonic generator can be activated to produce ultrasonic waves which can be transmitted from the distal end of the catheter(s) into the infection site to assist with debridement of the infection site. As described above, the ultrasonic waves can be applied to the infection site via a wire through the catheter(s), from a transducer disposed at the distal end of the catheter(s), or via the fluid within the catheter(s), as well as other pathways such as a separate catheter.

Finally, at block 309, the debris and fluid from the infection site can be cleared using the evacuation tube. The evacuation tube can include a check valve to prevent backflow of the fluid and debris. The evacuation tube can include a stop valve to pressurize the infection site. While fluid is being administered as described in block 303, the stop valve can prevent fluid from exiting the infection site to create pressure. Once the fluid has been administered and the ultrasonic waves have completed a cycle of debridement, the stop valve can be opened, and the fluid and debris drained from the infection site. In some embodiments, suction can be coupled to the evacuation tube to help clear the fluid and debris from the infection site. In some embodiments, the steps described in blocks 303 through 309 can be repeated until an infection is cleared.

Although the flowchart shows a specific order of execution, it is understood that the order of execution can differ from that which is depicted. For example, the order of execution of two or more blocks can be scrambled relative to the order shown. Also, two or more blocks shown in succession can be executed concurrently or with partial concurrence. Further, in some embodiments, one or more of the blocks shown in the flowchart can be skipped or omitted. It is understood that all such variations are within the scope of the present disclosure.

Now having described various embodiments of the present disclosure, additional features and combinations of features are described below.

Feature 1. The present disclosure provides for a method of using a debridement system, comprising: introducing at least a delivery tube and at least an evacuation tube into an infection site; administering fluid to the infection site via the delivery tube; applying ultrasonic waves to the infection site using an ultrasonic generator; and clearing debris from the infection site via the evacuation tube. Feature 2. The method of any combination of features, wherein the delivery tube and the evacuation tube are comprised in a catheter, wherein the catheter is introduced into the infection site. Feature 3. The method of any combination of features, wherein a first catheter includes the delivery tube and a second catheter includes the evacuation tube, wherein the first catheter and the second catheter are each introduced into the infection site. Feature 4. The method of any combination of features, wherein applying ultrasonic waves to the infection site comprises propagating ultrasonic waves via the first catheter, the second catheter or both the first catheter and the second catheter. Feature 5. The method of any combination of features, wherein applying ultrasonic waves to the infection site comprises propagating ultrasonic waves along one or more wires disposed within the first catheter, the second catheter or both the first catheter and the second catheter. Feature 6. The method of any combination of features, further comprising a third catheter for applying ultrasonic waves to the infection site. Feature 7. The method of any combination of features, wherein applying ultrasonic waves to the infection site comprises propagating ultrasonic waves along a wire disposed within the third catheter. Feature 8. The method of any combination of features, wherein applying ultrasonic waves to the infection site comprises propagating ultrasonic waves along a wire disposed in the catheter. Feature 9. The method of any combination of features, further comprising administering antibiotics to the infection site via the delivery tube. Feature 10. The method of any combination of features, wherein clearing debris from the infection site via the evacuation tube further comprises applying suction to the infection site via the evacuation tube. Feature 11. The method of any combination of features, wherein clearing debris from the infection site via the evacuation tube further comprises pressurizing the infection site with fluid from the delivery tube and a flow regulator on the evacuation tube. Feature 12. The method of any combination of features, wherein the fluid administered to the infection site comprises normal saline or hypertonic saline. Feature 13. The method of any combination of features, wherein applying ultrasonic waves to the infection site using an ultrasonic generator further comprises propagating ultrasonic waves along a wire disposed within the catheter.

Feature 14. A system comprising: at least a delivery tube and at least an evacuation tube; and an ultrasonic generator configured to cause ultrasonic waves to be emitted from a distal end of the catheter. Feature 15. The system of any combination of features, wherein the delivery tube and the evacuation tube are comprised in a catheter. Feature 16. The system of any combination of features, wherein a first catheter includes the delivery tube and a second catheter includes the evacuation tube. Feature 17. The system of any combination of features, wherein a wire is disposed within the first catheter, the second catheter or both the first catheter and the second catheter. Feature 18. The system of any combination of features, wherein the ultrasonic generator is disposed at the distal end of the catheter or is connected to a proximal end of the catheter. Feature 19. The system of any combination of features, further comprising a flow regulator at a distal end of the delivery tube. Feature 20. The system any combination of features, further comprising a flow regulator at a distal end of the evacuation tube. Feature 21. The system of any combination of features, wherein the flow regulator comprises a check valve. Feature 22. The system of any combination of features, further comprising a wire disposed within the catheter, the wire configured to transmit the ultrasonic waves from the ultrasonic generator to the distal end of the catheter. Feature 23 The system of any combination of features, wherein the evacuation tube is connected to a vacuum at a proximal end of the catheter, such that the evacuation tube provides suction at the distal end of the catheter. Feature 24. The system of any combination of features, further comprising a radiopaque marker disposed at the distal end of the catheter. Feature 25. The system of claim 14, wherein the catheter further comprises a hoop on an exterior side of the catheter, the hoop configured to receive a stitch to hold the catheter in place during a procedure. Feature 26. The system of any combination of features, wherein the catheter further comprises a depth gauge. Feature 27. The system of any combination of features, wherein the depth gauge comprises one or more markings disposed on an exterior surface of the catheter. Feature 28. The system of any combination of features, wherein the catheter has an outer diameter in a range of approximately 5 to 7 millimeters. Feature 29. The system of any combination of features, wherein at least one of the delivery tube or the evacuation tube has an inner diameter in a range of approximately 2 to 6 millimeters. Feature 30. The system of any combination of features, wherein the catheter is a pigtail catheter.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1 percent to about 5 percent” should be interpreted to include not only the explicitly recited concentration of about 0.1 weight percent to about 5 weight percent but also include individual concentrations (e.g., 1 percent, 2 percent, 3 percent, and 4 percent) and the sub-ranges (e.g., 0.5 percent, 1.1 percent, 2.2 percent, 3.3 percent, and 4.4 percent) within the indicated range. The term “about” can include traditional rounding according to significant figures of the numerical value. In addition, the phrase “about ‘x’ to ‘y’” includes “about ‘x’ to about ‘y’”.

Many variations and modifications may be made to the above-described aspects. All such modifications and variations are intended to be included herein within the scope of this disclosure and protected by the following claims.

Therefore, the following is claimed:

1. A method of using a debridement system, comprising:
introducing at least a delivery tube and at least an evacuation tube into an infection site;
administering fluid to the infection site via the delivery tube;
applying ultrasonic waves to the infection site using an ultrasonic generator; and
clearing debris from the infection site via the evacuation tube.

2. The method of claim 1, wherein the delivery tube and the evacuation tube are comprised in a catheter, wherein the catheter is introduced into the infection site.

3. The method of claim 1, wherein a first catheter includes the delivery tube and a second catheter includes the evacuation tube, wherein the first catheter and the second catheter are each introduced into the infection site.

4. The method of claim 3, wherein applying ultrasonic waves to the infection site comprises propagating ultrasonic waves via the first catheter, the second catheter or both the first catheter and the second catheter.

5. The method of claim 4, wherein applying ultrasonic waves to the infection site comprises propagating ultrasonic waves along one or more wires disposed within the first catheter, the second catheter or both the first catheter and the second catheter.

6. The method of claim 3, further comprising a third catheter for applying ultrasonic waves to the infection site.

7. The method of claim 6, wherein applying ultrasonic waves to the infection site comprises propagating ultrasonic waves along a wire disposed within the third catheter.

8. The method of claim 2, wherein applying ultrasonic waves to the infection site comprises propagating ultrasonic waves along a wire disposed in the catheter.

9. A system, comprising:
at least a delivery tube and at least an evacuation tube; and
an ultrasonic generator configured to cause ultrasonic waves to be emitted from a distal end of the catheter.

10. The system of claim 9, wherein the delivery tube and the evacuation tube are comprised in a catheter.

11. The system of claim 9, wherein a first catheter includes the delivery tube and a second catheter includes the evacuation tube.

12. The system of claim 11, wherein a wire is disposed within the first catheter, the second catheter or both the first catheter and the second catheter.

13. The system of claim 9, wherein the ultrasonic generator is disposed at the distal end of the catheter or is connected to a proximal end of the catheter.

14. The system of claim 9, further comprising a flow regulator at a distal end of the delivery tube.

15. The system of claim 9, further comprising a flow regulator at a distal end of the evacuation tube.

16. The system of claim 15, wherein the flow regulator comprises a check valve.

17. The system of claim 9, further comprising a wire disposed within the catheter, the wire configured to transmit the ultrasonic waves from the ultrasonic generator to the distal end of the catheter.

18. The system of claim 17, wherein the evacuation tube is connected to a vacuum at a proximal end of the catheter, such that the evacuation tube provides suction at the distal end of the catheter.

19. The system of claim 9, further comprising a radiopaque marker disposed at the distal end of the catheter.

20. The system of claim 9, wherein the catheter further comprises a hoop on an exterior side of the catheter, the hoop configured to receive a stitch to hold the catheter in place during a procedure.
